(19) United States
(12) Patent Application Publication
(10) Pub. No.: US 2017/0165847 A1
(43) Pub. Date: Jun. 15, 2017

(54) RECONFIGURABLE ROBOT ARCHITECTURE FOR MINIMALLY INVASIVE PROCEDURES

(71) Applicant: KONINKLIJKE PHILIPS N.V., EINDHOVEN (NL)

(72) Inventors: ALEKSANDRA POPOVIC, BOSTON, MA (US); DAVID NOONAN, NEW YORK, NY (US)

(21) Appl. No.: 15/323,758

(22) PCT Filed: Jul. 6, 2015
(86) PCT No.: PCT/IB2015/055090

§ 371 Date: Jan. 4, 2017

Related U.S. Application Data
(60) Provisional application No. 62/024,527, filed on Jul. 15, 2014.

(51) Int. Cl. B25J 18/00; A61B 34/30; B25J 18/02

(52) U.S. Cl. CPC B25J 18/005; B25J 18/007; B25J 18/025; A61B 34/30; A61B 2034/301

(57) ABSTRACT

A reconfigurable robot system employing a base actuator (11), an instrument actuator (12), an end-effector (13) and arm sets (14, 15). Each arm set (14, 15) is operable to successively adjoin the base actuator (11), the instrument actuator (12) and the end-effector (13) into an arc configuration for moving the instrument as held by the end-effector (13) relative to a remote center of motion responsive to the base actuator (11) generating the rotational motion along a primary axis and/or the instrument actuator (12) generating the rotational motion along a secondary axis. Each arc configuration defines the remote center of motion as an intersection of the primary axis, the secondary axis and the longitudinal axis. The arm sets (14, 15) are partially or fully interchangeable for reconfiguring the arc configuration of the base actuator (11), the instrument actuator (12) and the end-effector (13).

### FIELD OF THE INVENTION

The present disclosure generally relates to robots utilized during minimally invasive procedures (e.g., cardiac surgery, laparoscopic surgery, natural orifice transluminal surgery, pulmonary/bronchoscopy surgery and diagnostic interventions). The present disclosure specifically relates to a reconfigurable robot architecture adaptable to a correct range of motion during a specific minimally procedure.

### BACKGROUND OF THE INVENTION

Minimally invasive surgery is performed using elongated instruments inserted into the patient's body through small ports. The main visualization method during these procedures is an endoscope. In a standard workflow, the surgeon is holding two (2) surgical instruments while an operating room technician or a nurse are holding the endoscope. This setup can be uncomfortable, as the hands of the doctor and technician/nurse may be overlapped for the duration of procedure and the surgeon needs to continuously communicate endoscope motion to the technician/nurse. For this reason, one (1) or more instruments, including the endoscope, can be held by a robot that is controlled by the surgeon.

More particularly, the small ports that are placed on the patient's body are the only incision points through which the instruments may pass through to access the inside of the patient. As such, the instruments may be operated to rotate around these fulcrum points, but the instruments should not be operated in a manner that imposes translational forces on the ports to prevent any potential injury and harm to the patient. This is especially important for robotic guided surgery.

To the end, some known robots implement what is known as a remote center of motion (RCM) at the fulcrum point whereby a robot enforces an operating principle that only rotation of an instrument can be performed at a port and all translational forces of the instrument at that port are eliminated. This can be achieved by implementing a mechanical design which has a RCM at a specific location in space, and then aligning that point in space with the port. Alternatively, the RCM can be implemented virtually within the software of a robotic system, provided sufficient degrees of freedom exist to ensure the constraints of the RCM can be met.

As practiced, robotic systems have a predefined workspace. In minimally invasive surgery, this means that a specific robotic kinematics can be used only for those types of procedures where the required range of motion of the instrument is within the workspace. This presents a limitation not only on type of the procedure that a particular robot can perform, but also on size of the patient. Generally, to overcome this problem, conventional robotic systems are designed so that their workspace covers all intended uses of the robot. However, a large workspace results in larger robot components which further impacts overall size, weight, and may impact workflow as larger robot may collide with the environment. This problem is emphasized in already constrained environments, such as hybrid operating room, catheterization lab, or computed-tomography/magnetic resonance imaging systems.

### SUMMARY OF THE INVENTION

The present disclosure provides a reconfigurable robotic system adaptable to a desired range of motion of an instrument (e.g., an endoscope) during a specific minimally invasive procedure while maintaining minimal footprint and remote center of motion of the robot. The present disclosure further provides a method to select an appropriate workspace and appropriate orientation of the robot.

One form of the inventions of the present disclosure is a reconfigurable robot system employing a base actuator, an instrument actuator, an end-effector and a plurality of arm sets. The base actuator is operable to generate a rotational motion along a primary axis. The instrument actuator is operable to generate a rotational motion along a secondary axis. The end-effector is operable to hold the instrument along a longitudinal axis.

Each arm set is operable to successively adjoin the base actuator, the instrument actuator and the end-effector into an arc configuration for moving the instrument as held by the end-effector relative to a remote center of motion responsive to the base actuator generating the rotational motion along the primary axis and/or the instrument actuator generating the rotational motion along the secondary axis.

Each arc configuration defines the remote center of motion as an intersection of the primary axis, the secondary axis and the longitudinal axis, and

The arms sets are at least partially interchangeable for reconfiguring the arc configuration of the base actuator, the instrument actuator and the end-effector.

For purposes of the present disclosure,
(1) terms of the art including, but not limited to, “actuator”, “rotational motion”, “axis”, “end-effector”, “instrument”, “arm”, “remote center of motion” and “arc length” are to be interpreted as understood in the art of the present disclosure and as exemplary described herein,
(2) labels “base” and “instrument” for the term “actuator” distinguish different actuators as described and claimed herein without specifying or implying any additional limitation to the term “actuator”,
(3) labels “primary”, “secondary” and “longitudinal” for the term “axis” distinguish different axes as described and claimed herein without specifying or implying any additional limitation to the term “axis”,
(4) the term “arm set” broadly encompasses a support arm of a fixed length or a variable length being adjoined to or structurally configured to be adjoined to both actuators, and an instrument arm a fixed length or a variable length being adjoined to or structurally configured to be adjoined to the instrument actuator and the end-effector,
(5) labels “support” and “instrument” for the term “arm” distinguish different arms as described and claimed herein without specifying or implying any additional limitation to the term “arm”,
(6) the term “adjoin” in any tense broadly encompasses any type of affixation or detachable coupling of components involving direct physical contact between the components or an adjacent placements of the components, and
(7) the term “arc configuration” broadly encompasses a non-parallel angular orientation of the axes of the base actuator, the instrument actuator and the end-effector involving an base arc length between the base actuator and the instrument actuator and an extension arc length between the instrument actuator and the end-effector.
(8) labels “base” and “extension” for the term “arc length” distinguish different arms as described and claimed herein without specifying or implying any additional limitation to the term “arc length”, and
(9) the phrase “at least partially interchangeable” broadly encompasses each arm set being distinctive in terms of both arms being unique to the arm set, but possibly having each arm individually in common with one or more other arm sets whereby an interchange of one arm set for another arm set involves an exchange of one or both arms of the arms sets.

A second form of the inventions of the present disclosure is the reconfigurable robot system further employing a robot platform coupled to the base actuator to position (i.e., locate and/or orient) the end-effector as adjoined to the base actuator within a reference coordinate system (e.g., an operating table, a robot coordinate system or a patient coordinate system).

A third form of the inventions of the present disclosure is the reconfigurable robot system employing a robot configuration workstation operable to simulate a workspace relative to the remote center of motion for the instrument as held by the end-effector within each of the arc configurations of the base actuator, the instrument actuator and the end-effector and/or recommend one or more of the arm sets to be adjoined to the base actuator, the instrument actuator and the end-effector as a function of at least one of a specified pitch and a specified yaw of a workspace relative to the remote center of motion for the instrument as held by the end-effector.

For purposes of the present disclosure,
(1) terms of the art including, but not limited to, “robot platform”, “workstation”, “pitch range”, “yaw range”, and “workspace” are to be interpreted as understood in the art of the present disclosure and as exemplary described herein,
(2) examples of the term “workstation” include, but are not limited to, an assembly of one or more computing devices (e.g., a client computer, a desktop and a tablet), a display/monitor, and one or more input devices (e.g., a keyboard, joysticks and mouse),
(3) a structural configuration of a “computing device” may include, but is not limited to, processor(s), computer-usable/computer readable storage medium(s), an operating system, application module(s), peripheral device controller(s), slot(s) and port(s), and
(4) the term “application module” broadly encompasses a component of the workstation consisting of an electronic circuit and/or an executable program (e.g., executable software and/firmware) for executing a specific application.

A fourth form of the inventions of the present disclosure is each arm set employing an identification marker, particularly for identification purposes by the robot configuration workstation. Examples of an identification marker include, but are not limited to, markers involved in radio-frequency identification, near field communication, resistive or magnetic measurements, and optical encoding and measurement.

The foregoing forms and other forms of the inventions of the present disclosure as well as various features and advantages of the present disclosure will become further apparent from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

FIGS. 1A-1C illustrate exemplary embodiments of reconfigurable robot in accordance with the inventive principles of the present disclosure.

FIGS. 2A-2D illustrate exemplary embodiments of an arc arm in accordance with the inventive principles of the present invention.

FIG. 3 illustrates an exemplary embodiment of a reconfigurable robot system for minimally invasive procedures in accordance with the inventive principles of the present disclosure.

FIG. 4A illustrates a first exemplary embodiment of a concatenated robot for minimally invasive procedures in accordance with the inventive principles of the present disclosure.

FIG. 4B illustrates a second exemplary embodiment of a concatenated robot for minimally invasive procedures in accordance with the inventive principles of the present disclosure.

FIGS. 5A and 5B illustrate an exemplary adjoining of a proximal concentric arc and a distal concentric arc to an actuator in accordance with the inventive principles of the present disclosure.

FIG. 6 illustrates an exemplary coupling of a reconfigurable robot and configurable robot platform in accordance with the inventive principles of the present disclosure.

FIG. 7 illustrates a flowchart representative of an exemplary embodiment of a robot configuration simulation/recommendation method in accordance with the inventive principles of the present disclosure.

### DETAILED DESCRIPTION OF THE PREFERRED EMBODIMENTS

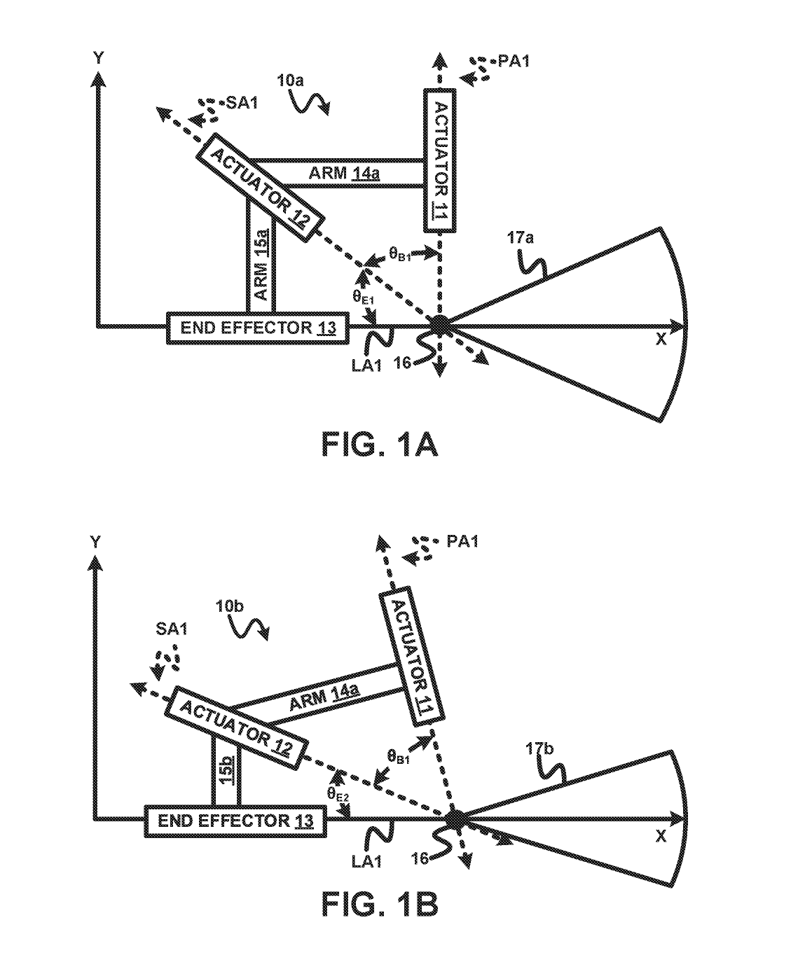

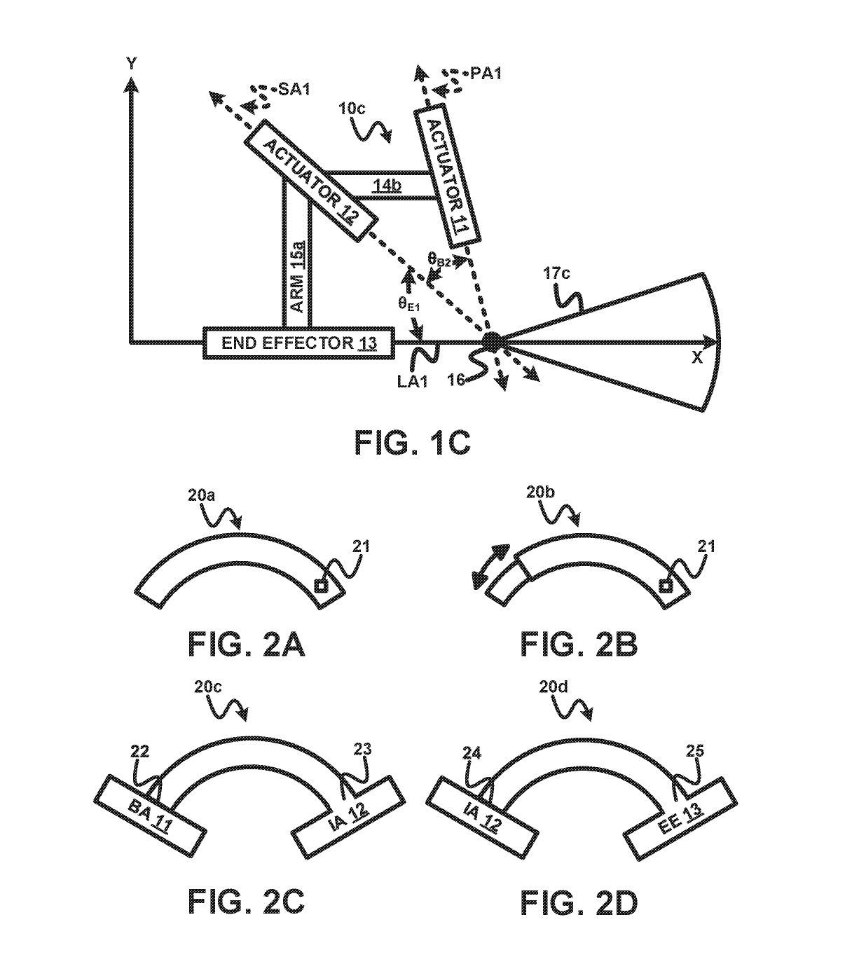

To facilitate an understanding of the present disclosure, the following description of FIGS. 1 and 2 teaches basic inventive principles of a reconfigurable robot system based on a reconfigurable robot having a remote center of motion established by an intersection of axes associated with actuators and end-effectors. From this description, those having ordinary skill in the art will appreciate how to apply the inventive principles of the present disclosure to any type of such reconfigurable robot for moving an instrument (e.g., an endoscope or other medical instrument, or a non-medical instrument) within a workspace relative to the remote center of motion.

Referring to FIGS. 1A and 1B, a reconfigurable robot of the present disclosure employs a base actuator 11, an instrument actuator 12 and an end effector 13.

Base actuator 11 as known in the art is selectively operated to generate a rotational motion along a primary axis PA1.

Instrument actuator 12 as known in the art is selectively operated to generate a rotation motion along a secondary axis SA1.

End-effector 13 as known in the art holds an instrument to be utilized during a minimally invasive procedure. The instrument is held along a longitudinal axis LA1 for facilitating an operation of the instrument. For example, an endoscope may be held by end-effector 13 whereby an axis of an insertion tube of an endoscope symbolized by the X axis is aligned with longitudinal axis LA1.

An intersection of primary axis PA1, secondary axis SA1 and longitudinal axis LA1 defines a remote center of motion 16. A distal section of the instrument extends from the remote center of motion 16 establishes a height of a workspace 17 having a conical shape. Workspace 17 has a pitch range relative to the Y axis and a yaw range relative to a Z axis (not shown) dependent upon a base arch length of θ₁₃ between primary axis PA1 and secondary axis SA1 and further dependent upon an extension arch length of θ_{E} between secondary axis SA1 and longitudinal axis LA1.

A reconfigurable robot of the present disclosure further employs a plurality of interchangeable arms sets for establishing different arc configurations, each having a base arch length of θ₁₃ between primary axis PA1 and secondary axis SA1 and an extension arch length of θ_{E} between secondary axis SA1 and longitudinal axis LA1.

For example, FIG. 1A illustrates an arm set including a support arm 14a and an instrument arm 15a. Support arm 14a is adjoined to base actuator 11 and instrument actuator 12, and instrument arm 15a is adjoined to instrument actuator 12 and end effector 13 to establish an arc configuration having a base arch length of θ_{B1} between primary axis PA1 and secondary axis SA1 and an extension arch length of θ_{E}1 between secondary axis SA1 and longitudinal axis LA1. A workspace 17a extending from remote center of motion 16 has a pitch range relative to the Y axis and a yaw range relative to a Z axis (not shown) dependent upon base arch length of θ_{B1} and extension arch length of θ_{E}1.

By further example, FIG. 1B illustrates an arm set including a support arm 14a and an instrument arm 15b. Support arm 14a is adjoined to base actuator 11 and instrument actuator 12, and instrument arm 15b is adjoined to instrument actuator 12 and end effector 13 to establish an arc configuration having base arch length of θ_{B1} between primary axis PA1 and secondary axis SA1 and an extension arch length of θ_{E}2 between secondary axis SA1 and longitudinal axis LA1. A workspace 17b extending from remote center of motion 16 has a pitch range relative to the Y axis and a yaw range relative to a Z axis (not shown) dependent upon base arch length of θ_{B1} and extension arch length of θ_{E2}.

By further example, FIG. 1C illustrates an arm set including a support arm 14b and an instrument arm 15a. Support arm 14b is adjoined to base actuator 11 and instrument actuator 12, and instrument arm 15a is adjoined to instrument actuator 12 and end effector 13 to establish an arc configuration having a base arch length of θ_{B2} between primary axis PA1 and secondary axis SA1 and extension arch length of θ_{E1} between secondary axis SA1 and longitudinal axis LA1. A workspace 17c extending from remote center of motion 16 has a pitch range relative to the Y axis and a yaw range relative to a Z axis (not shown) dependent upon base arch length of θ_{B2} and extension arch length of θ_{E}1.

Of importance to note is workspace 17a is larger than workspace 17b in view of a summation of base arch length of θ_{B1} and extension arch length of θ_{E1} being greater than a summation of base arch length of θ_{B1} and extension arch length of θ_{E1}, and is larger than workspace 17c due to a summation of base arch length of θ_{B1} and extension arch length of θ_{E1} being greater than a summation of base arch length of θ_{B2} and extension arch length of θ_{E1}. Further, workspace 17b and workspace 17c are the same size in view of a summation of base arch length of θ_{B1} and extension arch length of θ_{E2} being equal to a summation of base arch length of θ_{B2} and extension arch length of θ_{E1}.

The present disclosure is premised on the arm sets being interchangeable to thereby facilitate a selective increase or decrease in the pitch range and/or the yaw range of the workspace of the reconfigurable robot. To be interchangeable, in practice, support arms and/or instrument arms of the arms sets must be exchangeable. To this end, how each arm is adjoined to the actuators and end-effector determines the degree of interchangeability of the arm sets.

For example, the arm set of FIG. 1A and the arm set of FIG. 1B both employ the same support arm 14a, but different instrument arms 15a and 15b. To be interchangeable, support arm 14a is either affixed or detachably coupled to actuators 11 and 12, and each instrument arm 15a and 15b may be detachably coupled to actuator 12 and affixed or detachably coupled to end-effector 13. An exchange of instrument arm 15a as shown in FIG. 1A for instrument arm 15b as shown in FIG>1B involves a detachment of instrument arm 15a from actuator 12 and a detachable coupling of instrument arm 15b to actuator 12. For the exchange, an additional end-effector 13 is affixed with instrument arm 15b, or end-effector 13 is detached from instrument 15a and detachable coupled to instrument arm 15b.

Similarly for example, the arm set of FIG. 1A and the arm set of FIG. 1C both employ the same instrument arm 15a, but different support arms 14a and 14b. To be interchangeable, each support arm 14a and 14b may be detachably coupled to one or both actuators 11 and 12, and instrument arm 15a is affixed or detachably coupled to actuator 12 and end-effector 13. An exchange of support arm 14a as shown in FIG. 1A for support arm 14b as shown in FIG. 1B involves a detachment of support arm 14a from actuator 11 and a detachable coupling of support arm 14b to actuator 11. For the exchange, support arm 14b is detachably coupled to actuator 12 as still adjoined to end-effector 13 and instrument arm 15, or additional actuators 12, end-effector 13 and instrument arm 15a are affixed or detachably coupled to support arm 14b.

In practice, support arms 14 and instrument arms 15 may have any shape, may have a fixed or variable length, and may be adjoined at any angular orientation to actuators 11 and 12.

For example, FIG. 2A illustrates an arc arm 20a having a fixed arc length and FIG. 2B illustrates a telescoping arc arm 20b having a variable arc length symbolized by the bi-directional arrow. For each arc arm 20 and any other type of arm, an identification marker 21 may be employed as known in the art to identify the type of arm and/or a length of the arm when employed within a reconfigurable robot of the present disclosure. Examples of identification marker 21 include, but are not limited to, markers involved in radio-frequency identification, near field communication, resistive or magnetic measurements, and optical encoding and measurement.

By further example, FIG. 2C illustrates an oblique detachable coupling 22 of a support arc arm 20c to base actuator 11 and an oblique affixation 23 of support arc arm 20c to instrument actuator 12, and FIG. 2D illustrates an oblique detachable coupling 24 of an instrument arc arm 20d to instrument actuator 12 and an oblique affixation 25 of instrument arc arm 20d to end-effector 13.

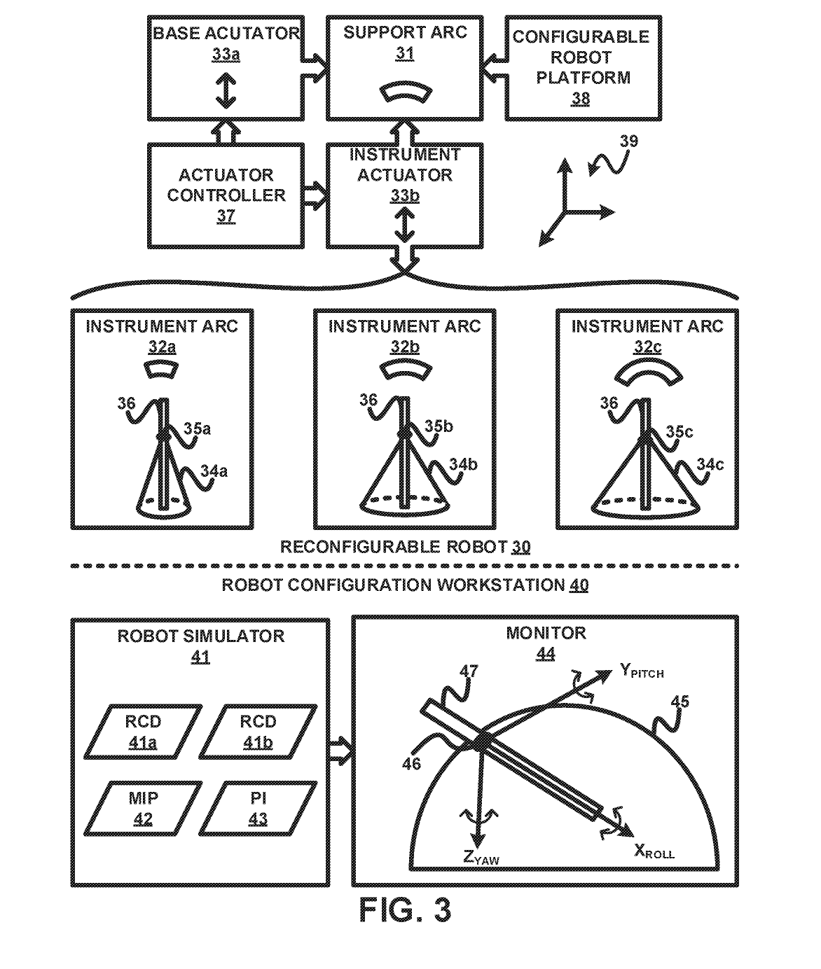

To further facilitate an understanding of the present disclosure, the following description of FIGS. 3-7 teaches the basic inventive principles of a recoverable robot system as shown in FIGS. 1 and 2 for a concatenated robot having a remote center of motion established by a concentric coupling of a support arc to one of a X number of instrument arcs, X≧2. From this description, those having ordinary skill in the art will appreciate how to apply the inventive principles of the present invention to any type of such concatenated robot as well as other types of robots for moving an instrument (e.g., an endoscope or other medical instrument, or a non-medical instrument) within a workspace relative to the remote center of motion.

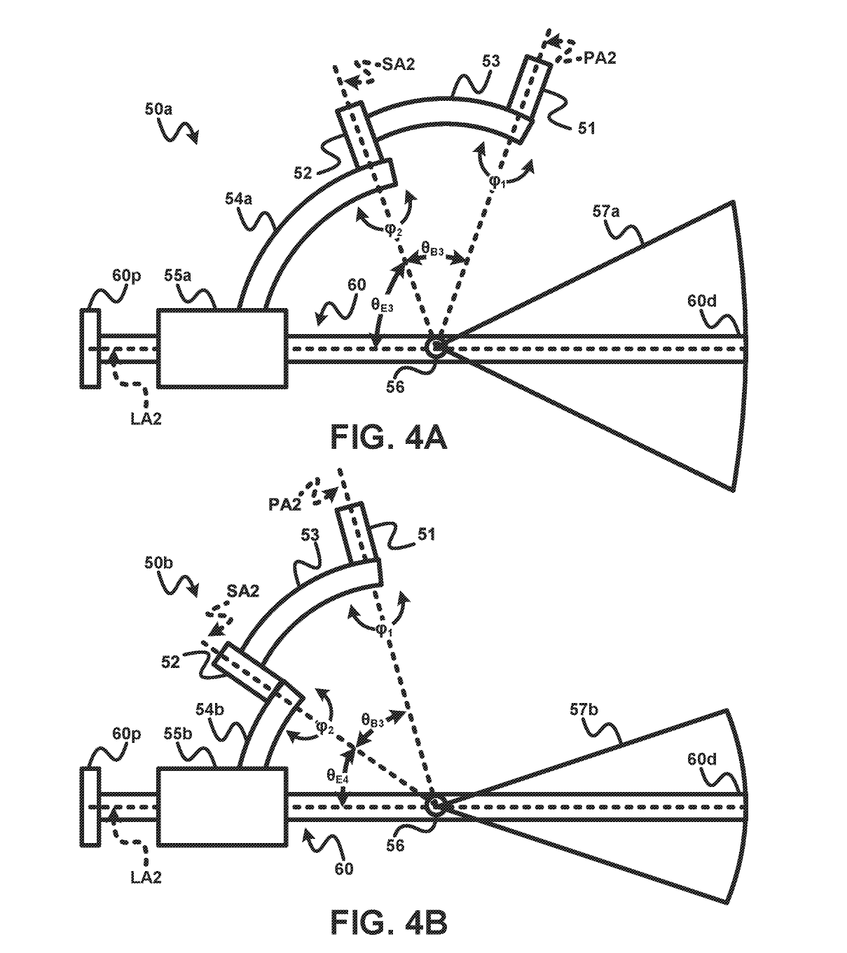

Referring to FIG. 3, a reconfigurable robot 50 employs a support arc 31 and three (3) instrument arcs 32 to configure a concatenated robot by concentrically coupling support arc 31 to one of the instrument arcs 32 via an actuator 33b. As will be further exemplary described herein in connection with FIG. 4, in operation, a actuator controller 37 as commanded selectively activates an actuator 33a to co-rotate support arc 31 and the concentrically coupled instrument arc 32 about a primary rotation axis as symbolized by the bi-directional arrow within actuator 33a. Concurrently or sequentially, actuator controller 37 as commanded selectively activates actuator 33b to rotate the concentrically coupled instrument arc 32 about a secondary rotation axis as symbolized by the bi-directional arrow within actuator 33b.

More particular to the arcs, support arc 31 has a base arc length as symbolized by the arc length therein, and each instrument arc 32 has a different extension arc length as symbolized by the different arcs length therein. As will be further exemplary described herein in connection with FIG. 4, the base arc length of support arc 31 and an extension arc length of the concentrically coupled instrument arc 32 co-establish a remote center of motion 35 defined by an intersection of the rotation axes of actuators 33. Based on remote center of motion 35, the concatenated robot defines a workspace 34a of an instrument 36 (e.g., an endoscope) adjoined via an affixation or detachable coupling of an end-effector (not shown) to instrument arc 32.

Specifically, workspace 34a encompasses a range of motion of a portion of the instrument 36 extending from the end effector (not shown) affixed to the concentrically coupled instrument arc 32 through remote center of motion 35. In practice for a minimally invasive procedure, surgical or diagnostic, a location of remote center of motion 35 coincides with a patient port as known in the art whereby workspace 34a facilitates pivoting/rotational motion of instrument 36 relative to remote center of motion 35 for purposes of the procedure that impedes, if not eliminates, any harm and damage to the patient. As such, workspace 34 will typically have a conical shape as shown in FIG. 3.

Of importance to the present disclosure, dimensions of a surface and a base of a conically shaped workspace 34 is dependent upon the base arc length of support arc 31 and the extension arc length of the corresponding instrument arc 32 as well as the length of the end-effector 39. For the concatenated robot, the base arc length of support arc 31 is fixed whereby the extension arc length of the corresponding instrument arc 32 becomes the predominant factor in a dimensioning of a surface and a base of the conically shaped workspace 34. As shown in FIG. 3, the dimensions of the surface and the base of the conically shaped workspace 34 increases as the extension arc length increases from instrument arc 32a to instrument arc 32c. For minimally invasive procedures, the differing extension arc lengths of instrument arcs 32 facilitate a recommendation or a selection of one or more of the instrument arcs 32 suitable for a particular minimally invasive procedure (e.g., thoracotomy, cardiac surgery, etc.) and/or a particular patient type (e.g., adult vs pediatric, a degree of any anorexia or any obesity, etc.)

To this end, a robot configuration workstation 40 employs a robot simulator 41 and a monitor 44. For purposes of the present disclosure, terms “workstation” and “monitor” are to be interpreted as understood in the art of the present disclosure and as exemplary described herein, and the term “robot simulator” broadly encompasses a component of a workstation consisting of an electronic circuit and/or an executable program (e.g., executable software and/firmware) for executing a specific application.

For workstation 40, robot simulator 41 implements a method for recommending or selecting one or more instrument arcs 32 suitable for a particular minimally invasive procedure and/or a particular patient type. To implement the method, robot simulator 41 processes concatenated robot data 41, minimally invasive procedure data 42 and patient data 43 received by and/or stored on workstation 40. As will be exemplary described herein in connection with FIG. 6, concatenated robot data 41 includes look-up tables organizing a relationship of numerous instrument arcs 32 of variable extension arc lengths to a particular base arc length of support arc 31 in terms of a “sufficient workspace” or an “insufficient workspace” for a designed range of instrument motion dependent upon a particular minimally invasive procedure and/or a particular patient type as indicated by data 42 and 43.

In practice, the concatenated robot may be coupled to a static robot platform (not shown) or a configurable robot platform 38 for selectively orienting workspace 34 relative to a reference coordinate system 39 (e.g., an operating table, a robot coordinate system or a patient coordinate system). Robot concatenated data 41a includes information of the arc lengths exclusive of any orientation information via any platform, and robot concatenated data 41b includes information of the arc lengths inclusive of orientation information via a platform 38.

From robot concatenated data 41b, robot simulator 41 generates a display on monitor 44 of a simulated anatomical region 45 having a port 46 and a simulated instrument 47 extending through port 46 into simulated anatomical region 45. In practice, simulated anatomical region 45 may be a graphical object as shown corresponding to the particular minimally invasive procedure and/or the particular patient type or a reconstructed image of the anatomical region, and the simulated instrument 47 may be a graphical object as shown corresponding to the particular minimally invasive procedure and/or the particular patient type or a standard image of instrument 47.

Robot simulator 41 enables a user-manipulation of simulated instrument 47 to select a desired range of motion of simulated instrument 47 in terms of a minimum pitch, a maximum pitch, a minimum yaw and a maximum yaw. Note a roll of simulated instrument 47 is not applicable to the workspace of simulated instrument 47.

In practice, based on the particular minimally invasive procedure and/or the particular patient information, robot simulator 41 may provide a default range of motion of simulated instrument 47 in terms of a minimum pitch, a maximum pitch, a minimum yaw and a maximum yaw that may be user-manipulated as desired.

Upon or during selection of the desired/default range of motion, robot simulator 41 accesses a look-up table associated with the desired range of motion whereby the look-up table will identify one or more instrument arcs 32 having extension arc length(s) for establishing an “sufficient workspace” with the base arc length of support arc 31 as will be exemplary described herein in connection with FIG. 6. The identified instrument arc(s) 32 are recommended or selected for concentric coupling to support arc 31 for achieving the desired workspace.

In practice, for embodiments incorporating configurable robot platform 38, robot simulator 41 identifies a middle point in the desired workspace in terms of (minimum pitch+maximum pitch)/2 and (minimum yaw+maximum yaw)/2 to obtain an orientation of the desired workspace.

To facilitate a further understanding of the inventive principles of the present disclosure, an exemplary reconfigurable robot system for moving an endoscope within a workspace relative to a remote center of motion will now be described herein in connection with FIGS. 4-7. For this example, the reconfigurable robot system has two (2) instrument arcs for configuring two (2) concatenated robots having different workspaces. From the description, those having ordinary skill in the art will appreciate how to incorporate additional instrument arcs.

Referring to FIG. 4A, a concatenated robot 50a employs actuator 51 having a primary axis PA2, an actuator 52 having a secondary axis SA2, a support arc 53, and an instrument arc 54a including an end-effector 55 for holding an endoscope 60 having a longitudinal axis LA2. Support arc 53 is concentrically connected to actuator 51 and actuator 52, and instrument arc 54a is concentrically connected to actuator 52. Of importance,
(1) rotational axes PA2, RAD and LA2 intersect at a remote center of motion 56,
(2) a base arch length of θ_{B} of support arc 53 extends between rotation axes PA2 and SA2,
(3) an extension arc length θ_{E3} of instrument arc 54a extends between rotation axes PA2 and LA2,
(4) a workspace 57a relative to remote center of motion 56 has surface and base dimensions derived from base arch length of θ_{B3} of support arc 53 and extension arc length θ_{E3} of instrument arc 54a,
(5) actuator 51 may be commanded to co-rotate arcs 53 and 54a about primary axis PA2 for a desired φ₁ degrees to control a broad movement of distal tip 60d of endoscope 60 within workspace 57a,
(6) actuator 52 may be commanded to rotate instrument arc 54a about secondary axis SA2 for a desired φ₂ degrees to control a targeted movement of distal tip 60d of endoscope 60 within workspace 57a, and
(7) end effector 55a has a capability, manual or controlled, of rotating endoscope 60 about its longitudinal axis LA2.

Referring to FIG. 4B, a concatenated robot 50b employs actuators 51 and 52, support arc 53, and an instrument arc 54b including an end-effector 55 for holding endoscope 60. Support arc 53 is concentrically connected to actuator 51 and actuator 52, and instrument arc 54b is concentrically connected to actuator 52. Of importance,
(1) rotational axes PA2, RAD and LA2 intersect at a remote center of motion 56,
(2) a base arch length of θ_{B} of support arc 53 extends between rotation axes PA2 and SA2,
(3) an extension arc length θ_{E4} of instrument arc 54b extends between rotation axes PA2 and LA2,
(4) a workspace 57b relative to remote center of motion 56 has surface and base dimensions derived from base arch length of θ_{B3} of support arc 53 and extension arc length θ_{E4} of instrument arc 54b,
(5) actuator 51 may be commanded to co-rotate arcs 53 and 54b about primary axis PA2 for a desired ₉₁ degrees to control a broad movement of distal tip 60d of endoscope 60 within workspace 57b,
(6) actuator 52 may be commanded to rotate instrument arc 54b about secondary axis SA2 for a desired ₉₂ degrees to control a targeted movement of distal tip 60d of endoscope 60 within workspace 57b, and
(7) end effector 55b has a capability, manual or controlled, of rotating endoscope 60 about its longitudinal axis LA2.

Referring to FIGS. 4A and 4B, the surface and base dimensions of workspace 57a are larger than the surface and base dimensions of workspace 57b due to extension arc length θ_{E3} of instrument arc 54a being larger than extension arc length θ_{E4} of instrument arc 34b. Consequently, while workspace 57a of concatenated robot 50a encircles workspaces 57b of concatenated robot 50b, each workspace 57 provides distinct advantages. For example, workspace 57a has a wider range of motion more suitable for anatomically open type of minimally invasive procedures while workspace 57b has a narrower range of motion more suitable for anatomically constricted type of minimally invasive procedures.

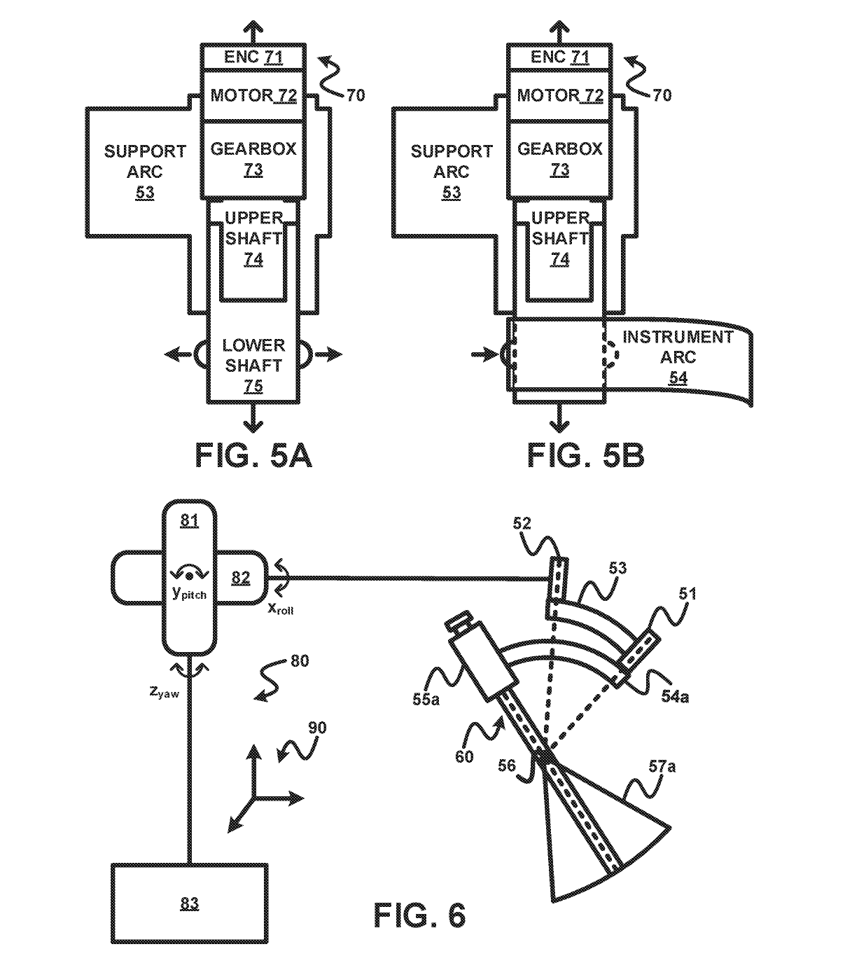

In practice, instrument arcs 54 may be coupled and decoupled to support arc 53 via actuator 52 in any manner known in the art. Referring to FIGS. 5A and 5B, an exemplary actuator 70 employs an encoder 71, a motor 72, a gearbox 73, an upper shaft 74 and a lower shaft 75 in vertical alignment establishing the secondary axis Symbolized by the vertical bi-directional arrow.

By commands from actuator controller 37, motor 71 provides rotational energy to gearbox 52 whereby upper shaft 72 and lower shaft 75 are rotated about the rotation axis. Support arc 53 encircles actuator 70 with lower shaft 75 downwardly extending from support arc 53. Instrument arc 34 slides onto lower shaft 75 and is secured thereto by snap-fits as shown, screws, magnets, clasps, or any other releasable mechanical coupling known in the art.

In practice, one (1) or more additional degrees of freedom may be added to concatenated robot 50a (FIG. 4A) or concatenated robot 50b (FIG. 4B) to orient the base of concatenated robots 50a and 50b to a predefined pitch angle and a predefined yaw angle relative to a reference coordinate system.

For example, refereeing to FIG. 6, a configurable robot platform 80 employs a pitch arm 81 and a yaw arm 82 connected to platform 83 to orient concatenated robot 50a relative to a predefined pitch angle and a predefined yaw angle relative to a reference coordinate system 70 (e.g., a coordinate system of platform 83, a coordinate system of concatenated robot 50a and operating table).

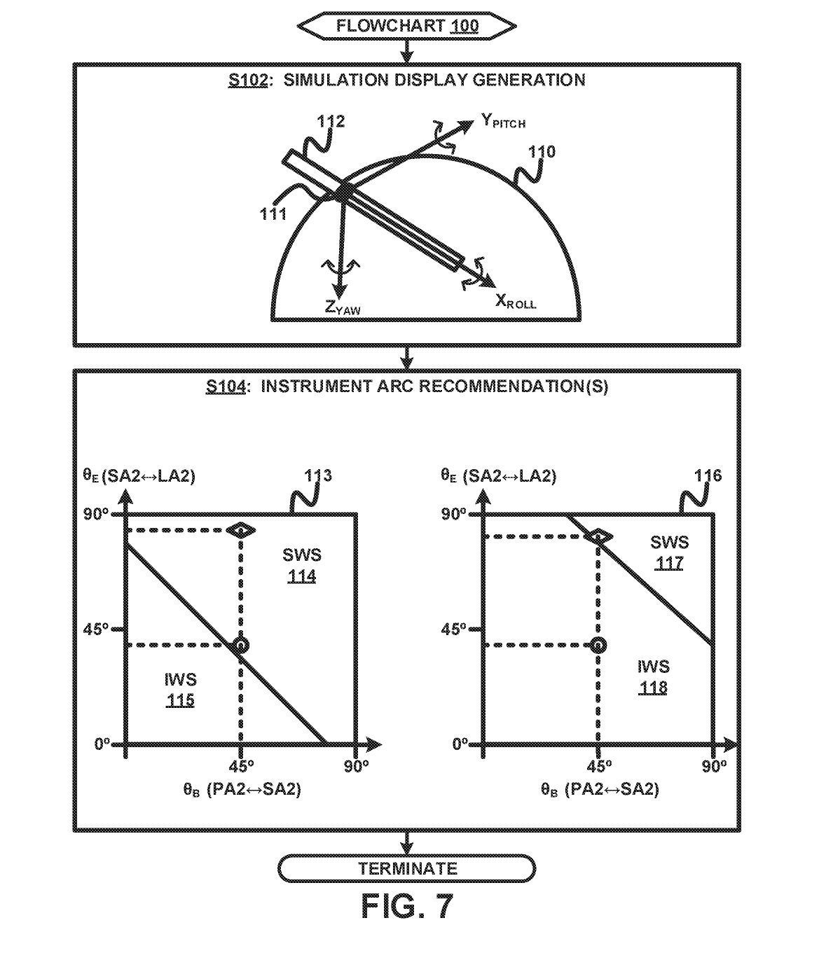

FIG. 7 illustrates a flowchart 100 representative of a robot configuration simulation method implemented by robot simulator 41 (FIG. 3) on behalf of concatenated robot 50a (FIG. 4A) or concatenated robot 50b (FIG. 4B).

Referring to FIG. 7, a stage S102 of flowchart 100 encompasses robot simulator 41 (FIG. 3) generating a simulation display on endoscope 60 (FIG. 4) within an anatomical region based on a particular minimally invasive procedure and/or a particular patient type. For example, as shown in stage S102, robot simulator 41 may generate a simulated anatomical region 110 having a port 111 and a simulated endoscope 112 extending through port 111 into simulated anatomical region 110 as previously described herein in connection with FIG. 3.

Stage S102 enables user-manipulation of simulated endoscope 112 to select a desired/default range of motion of simulated endoscope 112 in terms of a minimum pitch, a maximum pitch, a minimum yaw and a maximum yaw.

A stage S104 of flowchart 100 encompasses robot simulator 41 recommending either instrument arc 56a (FIG. 4A) and/or instrument arc 56b (FIG. 4B) or neither as indicated by a lookup table corresponding to the selected desired/default range of motion.

For example, as shown in stage S104, a lookup table 113 organizes various pairings of a base arc length θ_{B} and extension arc length θ_{E}, both lengths having a range [0°<, ≦90°] for a desired range of motion of [−20°, +20°] of yaw and [−50°, +50°] of pitch. Each pairing is classified as either “sufficient workspace” region 114 or “insufficient workspace” region 115. For this example, base arc length θ_{B} of support arc 33 (FIG. 2) is 45°, extension arc length θ_{E1} for instrument arc 56a is 85° and extension arc length θ_{E2} for instrument arc 56a is 40° . As such, a pairing of support arc 53 and instrument arc 56a is located within “sufficient workspace” region 114 as symbolized by the diamond within region 114, and a pairing of support arc 53 and instrument arc 56b is also located within “sufficient workspace” region 114 as symbolized by the circle within region 114. Robot simulator 41 therefore would recommend both instrument arc 46a and instrument arc 46b for concentric coupling to support arc 43.

By further example, as shown in stage S104, a lookup table 116 organizes various pairings of a base arc length θ_{B} and extension arc length θ_{E}, both lengths having a range [0°<, <90°] for a desired range of motion of [−50°, +50°] of yaw and [−50°, +50°] of pitch. Each pairing is classified as either “sufficient workspace” region 117 or “insufficient workspace” region 118. Again for this example, base arc length θ_{B} of support arc 33 (FIG. 2) is 45°, extension arc length θ_{E1} for instrument arc 56a is 85° and extension arc length θ_{E2} for instrument arc 56a is 40°. As such, a pairing of support arc 53 and instrument arc 56a is located within “sufficient workspace” region 117 as symbolized by the diamond within region 117, and a pairing of support arc 53 and instrument arc 56b is also located within “insufficient workspace” region 118 as symbolized by the circle within region 118. Robot simulator 41 therefore would only recommend instrument arc 56a concentric coupling to support arc 53.

In practice, robot simulator 41 includes numerous lookup table associated with various range of motion with the actual number of lookup tables dependent upon a desired degree of accuracy.

Also as shown by tables 113 and 116, the “sufficient workspace” region decreases as the range of motion increases across the lookup tables. Thus, the number of support arc/instrument arc pairings within the “sufficient workspace” regions also decrease as the range of motion in terms of yaw degrees and pitch degrees increase across the lookup tables. For any given whereby none of the possible support arc/instrument arc pairings are within a “sufficient workspace” region, robot simulator 41 will recommend the best reachable workspace on the simulated display.

Upon termination of flowchart 100, the recommended or best instrument arc may be concentrically coupled to the support arc for performing the minimally invasive procedure.

Referring to FIGS. 1-7, those having ordinary skill in the art will appreciate numerous benefits of the present disclosure including, but not limited to, a reconfigurable robotic system adaptable to a desired range of motion of an instrument (e.g., an endoscope) during a specific minimally invasive procedure while maintaining minimal footprint and remote center of motion of the robot.

Furthermore, as one having ordinary skill in the art will appreciate in view of the teachings provided herein, features, elements, components, etc. described in the present disclosure/specification and/or depicted in the FIGS. 1-7 may be implemented in various combinations of electronic components/circuitry, hardware, executable software and executable firmware and provide functions which may be combined in a single element or multiple elements. For example, the functions of the various features, elements, components, etc. shown/illustrated/depicted in the FIGS. 1-7 can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk read only memory (CD-ROM), compact disk read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive reconfigurable robot architecture for minimally invasive procedures, (which embodiments are intended to be illustrative and not limiting), it is noted that modifications and variations can be made by persons having ordinary skill in the art in light of the teachings provided herein, including the FIGS. 1-7. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A reconfigurable robot system, comprising:
a base actuator operable to generate a rotational motion along a primary axis;
an instrument actuator operable to generate a rotational motion along a secondary axis;
an end-effector operable to hold an instrument along a longitudinal axis; and
a plurality of arm sets,
wherein each arm set is operable to successively adjoin the base actuator, the instrument actuator and the end-effector into an arc configuration for moving the instrument as held by the end-effector relative to a remote center of motion responsive to at least one of the base actuator generating the rotational motion along the primary axis and the instrument actuator generating the rotational motion along the secondary axis,
wherein each arc configuration defines the remote center of motion as an intersection of the primary axis, the secondary axis and the longitudinal axis, and
wherein the arm sets are at least partially interchangeable for reconfiguring the arc configuration of the base actuator the instrument actuator and the end-effector.

2. The reconfigurable robot system of clam 1, further comprising:
a robot platform operable to be coupled to the base actuator to position the end-effector as adjoined to the base actuator within a reference coordinate system.

3. The reconfigurable robot system of claim 1, wherein each arm set includes a same support arm operable to be adjoined to the base actuator and the instrument actuator with a fixed length or a variable length between the base actuator and the instrument actuator.

4. The reconfigurable robot system of claim 1, wherein each arm set includes a different support arm operable to be adjoined to the base actuator and the instrument actuator with a fixed length or a variable length between the base actuator and the instrument actuator.

5. The reconfigurable robot system of claim 1, wherein each arm set; includes a same instrument arm operable to be adjoined to the instrument actuator and the end effector with a fixed length or a variable length between the instrument actuator and the end effector.

6. The reconfigurable robot system of claim 1, wherein each arm set includes a different instrument arm operable to be adjoined to the instrument actuator and the end effector with a fixed length or a variable length between the instrument actuator and the end effector.

7. The reconfigurable robot system of claim 1,
wherein each arm set includes a same support arm affixed with the base actuator and the instrument actuator.

8. The reconfigurable robot system of claim 1,
wherein each arm set includes a same support arm affixed with the instrument actuator; and
wherein each arm set further includes a different instrument arm operable to be detachably coupled to the instrument actuator.

9. The reconfigurable robot system of claim 1,
wherein each arm set includes an instrument arm; and
wherein the end-effector is operable to be detachably couple to each instrument arm.

10. The reconfigurable robot system of claim 1, wherein each arm set includes arms having an arc shape.

11. The reconfigurable robot system of claim 1, further comprising:
a robot configuration workstation operable to simulate a workspace relative to the remote center of motion for the instrument as held by the end-effector each of the arc configurations of the base actuator, the instrument actuator and the end-effector.

12. The reconfigurable robot system of claim 1, further comprising:
a robot configuration workstation operable to recommend at least one of the arm sets to be adjoined to the base actuator, the instrument actuator and the end-effector as a function of at least one of a specified pitch range and a specified yaw range of a workspace relative to the remote center of motion for the instrument as held by the end-effector

13. The reconfigurable robot system of claim 1, wherein each arm set includes arms structurally configured to provide a different pitch range and a different yaw range of a workspace for the instrument relative to the remote center of motion.

14. The reconfigurable robot system of claim 1, wherein each arm set includes an identification marker.

15. A reconfigurable robot, comprising:
a base actuator operable to generate a rotational motion along a primary axis;
an instrument actuator operable to generate a rotational motion along a secondary axis;
an end-effector operable to hold an instrument along a longitudinal axis;
a first arm set successively adjoining the base actuator, the instrument actuator and the end-effector into a first arc configuration for moving the instrument as held by the end-effector relative to a remote center of motion responsive to at least one of the base actuator generating the rotational motion along the primary axis and the instrument actuator generating the rotational motion along the secondary axis;
a second arm set operable to successively adjoin the base actuator, the instrument actuator and the end-effector into a second arc configuration for moving the instrument as held by the end-effector relative to the remote center of motion responsive to at least one of the base actuator generating the rotational motion along the primary axis and the instrument actuator generating the rotational motion along the secondary axis;
wherein the first arc configuration and the second arc configuration define the remote center of motion as an intersection of the primary axis, the secondary axis and the longitudinal axis;
wherein the first arm set and the second arm set are at least partially interchangeable for reconfiguring the first arc configuration of the base actuator, the instrument actuator and the end-effector to the second arc configuration of the base actuator, the instrument actuator and the end-effector; and
a robot configuration workstation operable to recommend at least one of the first arm set and the second art set to be adjoined to the base actuator, the instrument actuator and the end effector as a function of at least one of a specified pitch range and a specified yaw range of a workspace relative to the remote center of motion for the instrument as held by the end effector.

16. (canceled)

17. (canceled)

18. (canceled)

19. (canceled)

20. (canceled)
